(19) United States
(12) Patent Application Publication
(10) Pub. No.: US 2025/0155443 A1
(43) Pub. Date: May 15, 2025

(54) ALPHA-1 ANTITRYPSIN BIOMARKERS FOR DETECTING AND MONITORING CANCERS

(71) Applicant: Sun Jet Biotechnology Inc., Taipei City (TW)

(72) Inventor: Yeou-Guang Tsay, Taipei (TW)

(73) Assignee: Sun Jet Biotechnology Inc., Taipei City (TW)

(21) Appl. No.: 18/937,792

(22) Filed: Nov. 5, 2024

Related U.S. Application Data
(60) Provisional application No. 63/597,490, filed on Nov. 9, 2023.

(51) Int. Cl. G01N 33/574

(52) U.S. Cl. CPC G01N 33/57488; G01N 2333/8125

(57) ABSTRACT

Disclosed is a method for diagnosing a cancer health state, or a change in cancer health state in a patient, or for diagnosing a risk of the change or presence of a cancer in a patient, comprising determining, in a plasma sample from said patient, one or more biomarker values that correspond to alpha-1 antitrypsin (A1AT)-containing complex structures, and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

### CROSS REFERENCE

This non-provisional application claims the priority under 35 U.S.C. § 119(e) on U.S. Patent Provisional Application No. 63/597,490 filed on Nov. 9, 2023, the entire contents of which are hereby incorporated by reference.

### FIELD OF THE INVENTION

The present invention pertains to a biomarker and a use of biomarker based on alpha-1 antitrypsin (A1AT)-containing complex structures for detecting and monitoring cancers.

### BACKGROUND OF THE INVENTION

Hepatocellular carcinoma (HCC) is the most common type of primary liver cancer, accounting for approximately 80% of cases. HCC has a strong male predominance, with a male-to-female ratio estimated to be between 2:1 and 4:1. So far, alpha-fetoprotein (AFP) is the only cancer biomarker widely used in HCC screening. The overall sensitivity of AFP for HCC is approximately 70% of disease at all stages. However, it is not optimal for detecting early-stage HCC with small-sized tumors.

Ovarian cancer (OC) is the third most common gynecologic cancer with poor prognosis and the highest mortality rate. Ovarian cancer is often dubbed a silent killer due to having various symptoms that often only develop when the disease reaches an incurable stage. Currently, CA125 and human epididymis protein 4 (HE4) are the only two markers approved by the FDA for monitoring treatment and detecting disease recurrence for ovarian cancer. While HE4 has a limited role, CA125 has a sensitivity of 55% of stage I and II ovarian cancer.

Breast cancer (BC) is the second most common cancer for women, with approximately 2.5% of these cases resulting in death. Despite a high cure rate for localized disease, only 20% of BC cases are diagnosed at an early stage. International studies have reported that the biomarkers CA15-3 and CA27.29 could be used for BC screening, with sensitivities of 30%-57% and 55%-62%, respectively. For the Taiwanese population, the sensitivity of these two markers is quite low, at 5.5% and 6.4% each.

Overall, there is still a pressing need for biomarkers with better all-around performance in HCC, BC and OC screening. Protein complexes are the bona fide structures that harbor biological functions. In traditional western blotting, these protein complexes are difficult to be accurately quantified due to discrepancy in the electrotransfer of proteins with distinct sizes.

### SUMMARY OF THE INVENTION

The inventors surprisingly found that different species of alpha-1 antitrypsin (A1AT) complex structures (or multimeric structures) can be detected and quantified from human plasma sample, and that their levels and ratios are indicative of the risk of cancers including hepatocellular carcinoma (HCC), ovarian cancer (OC), and breast cancer (BC).

In one aspect, the present invention provides a method for diagnosing a cancer health state in a patient, comprising: determining, in a plasma sample from said patient, one or more biomarker values that correspond to alpha-1 antitrypsin (A1AT)-containing complex structures; and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

In one further aspect, the present invention provides a method for diagnosing a change in cancer health state in a patient, comprising: determining, in a plasma sample from said patient, one or more biomarker values that correspond to alpha-1 antitrypsin (A1AT)-containing complex structures; and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

In one yet aspect, the present invention provides a method for diagnosing a risk of the change or presence of a cancer in a patient, comprising: detecting, in a plasma sample from said patient, one or more biomarker values that correspond to alpha-1 antitrypsin (A1AT)-containing complex structures; and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

In one further aspect, the present invention provides use of an alpha-1 antitrypsin (A1AT)-containing complex structure as a biomarker for a cancer selected from the group consisting of hepatocellular carcinoma (HCC), ovarian cancer (OC), and breast cancer (BC).

In one yet aspect, the present invention provides a capture reagent for alpha-1 antitrypsin (A1AT) or an A1AT-containing complex structure, for use in diagnosing (in vitro) a cancer health state in a patient, wherein said cancer is selected from the group consisting of hepatocellular carcinoma (HCC), ovarian cancer (OC), and breast cancer (BC).

Specifically, the use comprising determining one or more biomarker values that correspond to A1AT-containing complex structures using the capture reagent for A1AT or A1AT-containing complex structure.

Also provided is a kit for performing the method as described herein, comprising a capture reagent for A1AT or an A1AT-containing complex structure, and instructions for performing the method.

In some embodiments, the one or more biomarker values are determined by performing an in vitro assay. The in vitro assay may be an immunoassay, including but not limited to a western blotting and a capillary electrophoresis.

In some embodiments, determining the biomarker values comprises performing an in vitro assay, wherein said in vitro assay comprises a capture reagent for A1AT or an A1AT-containing complex structure.

In some embodiments, the capture reagent is an antibody.

In some embodiments, the one or more biomarker values are determined by performing a capillary electrophoresis under non-reducing conditions. According to certain preferred embodiments, the one or more biomarker values include IP₅₈, IP₁₃₀, IP₁₈₀, or a combination thereof. According to certain preferred embodiments, the assigning is based on a ratio of said biomarker values selected from the group consisting of

$$\frac{I {P}_{130}}{I {P}_{58}} , \frac{I {P}_{180}}{I {P}_{58}} , and \frac{I {P}_{130} + I {P}_{180}}{I {P}_{58}} .$$

It is to be understood that both the foregoing general description and the following detailed description are exemplary and explanatory only and are not restrictive of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the results of western blotting for analyzing plasma A1AT species from three healthy subjects (male) and three hepatocellular carcinoma patients under reducing or non-reducing conditions. The left numbers indicate molecular mass markers (kDa).

FIG. 2 shows the migration profiles of plasma A1AT species in capillary electrophoreses for 9 healthy subjects (male), 28 healthy subjects (female), 53 hepatocellular carcinoma patients (male), 9 hepatocellular carcinoma patients (female), 51 ovarian cancer patients, and 161 breast cancer patients. All of the patients have cancers no more severe than stage 2. The left numbers indicate signal percentages relative to the ˜60-kDa peak, while the bottom numbers denote the migration positions of proteins corresponding to their molecular masses. The pair of numbers at the top of the shaded box, that contains P₅₈, P₁₃₀, or P₁₈₀, indicates the mass range boundaries used for summing signals, such as area P₅₈ primarily representing the A1AT monomer. The immunoblot on the right shows how the peaks in capillary western analyses match the structures resolved in SDS-PAGE.

FIG. 3 shows the box plot illustrating the statistical analysis of the A1AT indices in patients with hepatocellular carcinoma (male), hepatocellular carcinoma (female), ovarian cancer and breast cancer. The statistical analyses of three A1AT multimeric indices were performed. Each dot in the plot represents the index value for an individual subject, with the mean indicated for each group.

FIG. 4 shows the receiver operating characteristic (ROC) curves of the three A1AT indices distinguishing between healthy individuals and patients of hepatocellular carcinoma (male), hepatocellular carcinoma (female), ovarian cancer, and breast cancer. Sensitivity is represented on the vertical axis, and (1−specificity) is shown on the horizontal axis for each A1AT index cutoff value. Each dot in the plot corresponds to the point with the maximum (sensitivity+specificity). The specific cutoff values are marked by dots, with corresponding performance indicated using dotted lines aligning with the axes.

FIG. 5 shows a summary of the results of two indices for all healthy subjects (HS) and hepatocellular carcinoma (HCC) patients in the present embodiment. Dark grey shading indicates values that are two times higher than the cutoff, whereas light grey shading represents values falling between 1× and 2× the cutoff.

FIG. 6 shows a summary of the results of two indices for all female healthy subjects (HS) and ovarian cancer (OC) patients in the present embodiment. Dark grey shading indicates values that are two times higher than the cutoff, whereas light grey shading represents values falling between 1× and 2× the cutoff.

FIG. 7 shows a summary of the results of two indices for all female healthy subjects (HS) and breast cancer (BC) patients in the present embodiment. Dark grey shading indicates values that are two times higher than the cutoff, whereas light grey shading represents values falling between 1× and 2× the cutoff.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereto known to those skilled in the art.

The term “biomarker” used herein refers to a measurable characteristic, either within or external to an organism, that indicates a specific physiological state or the presence of a disease. Biomarkers can be used as indicators for assessing physiological processes, disease progression, drug response, or treatment effectiveness. They may include molecules, cells, tissues, physiological indicators, or imaging features, with their changes often closely associated with disease occurrence, progression, treatment response, etc. Biomarkers have significant applications in clinical diagnosis, prediction, monitoring, and treatment, aiding in improving the accuracy of early disease detection, diagnosis, prognosis assessment, as well as evaluating the effectiveness and safety of treatment regimens.

The term “cancer” used herein refers to a group of diseases characterized by the uncontrolled growth and spread of abnormal cells. These cells can invade and destroy surrounding healthy tissues and can also metastasize to distant parts of the body. Cancer can arise from almost any type of cell in the body and may develop in various organs and tissues. It is typically caused by genetic mutations or other factors that disrupt the normal regulation of cell growth and division.

A biomarker value for the biomarkers described herein can be determined using any of a variety of known analytical methods. In some embodiments, the biomarker value can be determined through performing an in vitro assay, for example, an immunoassay. In one embodiment, the determination of a biomarker value involves the use of a capture reagent. A biomarker value may also refer to a ratio calculated based on two or more biomarker values, e.g.,

$$\frac{I {P}_{130}}{I {P}_{58}} , \frac{I {P}_{180}}{I {P}_{58}} , and \frac{I {P}_{130} + I {P}_{180}}{I {P}_{58}} .$$

As used herein, a “capture agent” or “capture reagent” refers to a molecule that is capable of binding specifically to a biomarker. Capture reagents include but are not limited to aptamers, antibodies, antigens, adnectins, ankyrins, other antibody mimetics and other protein scaffolds, autoantibodies, chimeras, small molecules, an F(ab′)₂ fragment, a single chain antibody fragment, an Fv fragment, a single chain Fv fragment, a nucleic acid, a lectin, a ligand-binding receptor, affibodies, nanobodies, imprinted polymers, avimers, peptidomimetics, a hormone receptor, a cytokine receptor, and synthetic receptors, and modifications and fragments of these.

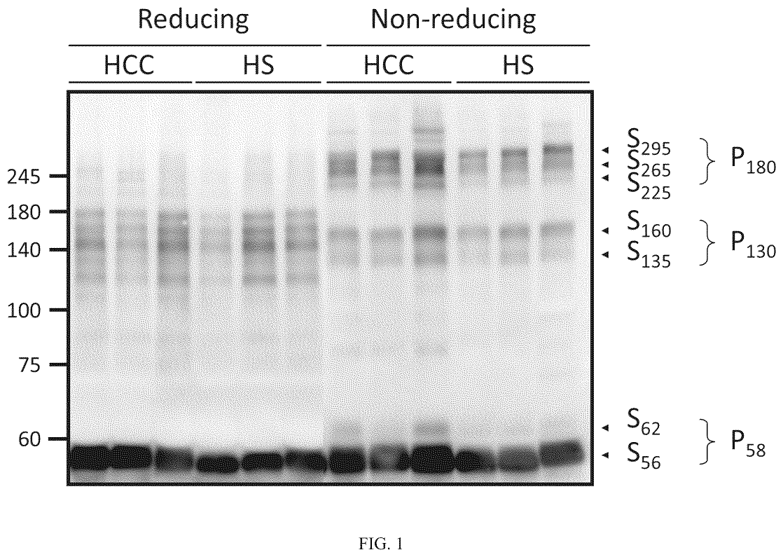

Alpha-1 antitrypsin, A1AT protein, is encoded by the SERPINA1 gene in humans and belongs to the serpin superfamily. According to the BioGPS database, this protein is expressed in various human tissues, including lung, small intestine, bone marrow, and liver. A1AT deficiency is well documented for its link to an increased risk of liver disease (Teckman J H, Jain A (2014) Advances in alpha-1-antitrypsin deficiency liver disease. Curr Gastroenterol Rep 16(1):367). For patients expressing defective A1AT protein, there is a tendency for A1AT mutants to accumulate within liver cells and undergo polymerization. These polymers are supposedly formed through non-covalent bonds and can dissociate into monomers even in the absence of reducing agents (Lomas D A, Evans D L, Finch J T, Carrell R W. (1992) The mechanism of Z alpha 1-antitrypsin accumulation in the liver. Nature. 357(6379):605). In contrast, our discovery is about A1AT entities with unique sizes only observed under non-reducing conditions and disappearing upon thiol-mediated reduction (FIG. 1). Thus, these circulating A1AT multimers are likely assembled through disulfide linkages. Also, while our findings reveal an association between A1AT complex formation and cancers, there was no past report about how these A1AT multimers may be related to any disease or health condition.

The terms “A1AT multimer,” “A1AT-containing complex structure,” and “A1AT complex structure” are used interchangeably herein and refer to a protein complex comprising at least one alpha-1 antitrypsin (A1AT) subunit, wherein the at least one A1AT subunit may be linked to one or more partners (proteins or polypeptides other than A1AT).

In one aspect, the present invention provides a method for diagnosing a cancer health state, or a change in cancer health state in a patient, or for diagnosing a risk of the change or presence of a cancer in a patient, comprising determining, in a plasma sample from said patient, one or more biomarker values that correspond to A1AT-containing complex structures, and assigning the patient as having or not having cancer, or having or not having a change in cancer health state, or having or not having a risk of cancer based on said biomarker values, wherein said cancer is selected from the group consisting of hepatocellular carcinoma (HCC), ovarian cancer (OC), and breast cancer (BC).

The one or more biomarker values may include or not include a biomarker value that corresponds to an A1AT monomer.

In another aspect, the present invention provides use of an A1AT-containing complex structure as a biomarker for a cancer selected from the group consisting of HCC, OC, and BC.

The present invention also provides a capture reagent for A1AT or an A1AT-containing complex structure, for use in diagnosing (in vitro) a cancer health state in a patient, wherein said cancer is selected from the group consisting of HCC, OC, and BC. Said use may comprise determining one or more biomarker values that correspond to A1AT-containing complex structures using the capture reagent for A1AT or A1AT-containing complex structure.

In one further aspect, the present invention provides a kit for performing the method as described herein, comprising a capture reagent for A1AT or an A1AT-containing complex structure, and instructions for performing the method.

In one further aspect, the present invention provides use of a capture reagent for A1AT or an A1AT-containing complex structure in the preparation of a kit for performing the method as described herein.

According to the present invention, a patient may be assigned as having or not having HCC, OC or BC, or having or not having a change in HCC, OC or BC health state, or having or not having a risk of HCC, OC or BC, based on a higher biomarker value that corresponds to an A1AT-containing complex structure, or a lower biomarker value that corresponds to an A1AT-containing complex structure.

As used herein, a higher (biomarker) value or lower (biomarker) value can refer to a value that is higher or lower compared with a reference level. For example, a lower value can be lower than a reference level by at least 30%, 40%, 50%, 60%, 70%, 80%, 90% or 100%; and higher value can be higher than a reference level by at least 30%, 40%, 50%, 60%, 70%, 80%, 90% or 100%. In some embodiments, a reference level can be a standard (or a threshold) value in a normal individual or a control group. For example, a standard or threshold value can be set based on an average or median level obtained from a cohort of normal subjects. In some embodiments, the cohort of subjects can be a population of normal human (without cancer, or without HCC, OC or BC). In addition, a threshold value can be set further based a desired sensitivity and/or specificity for detecting or diagnosing HCC, OC or BC.

According to certain embodiments of the present invention, four species or groups of human plasma A1AT-containing proteins can be resolved using conventional SDS-PAGE and western blotting under non-reducing conditions, including: (i) a 56-kDa monomeric species, (ii) a 62-kDa species, migrating slightly slower than the A1AT monomer, (iii) a 150-kDa group having two protein bands at around 135 and around 160 kDa, and (iv) a 260-kDa group having three polypeptides of molecular sizes of about 225, 265 and 295 kDa, respectively.

According to the present invention, human plasma A1AT complex structures can also be resolved by a capillary gel electrophoresis into three major peaks, including P₅₈, P₁₃₀, and P₁₈₀, and correspond to the 56/62-kDa species, the 150-kDa group, and the 260-kDa group, respectively.

A biomarker value is indicative of a concentration of a biomarker, or a ratio of concentrations of the biomarkers in a sample. A biomarker value of the present invention may be a signal intensity or normalized signal intensity of any of the peaks P₅₈, P₁₃₀, and P₁₈₀, denoted as IP₅₈, IP₁₃₀, and IP₁₈₀, respectively, or a ratio of the signal intensities. The signal intensity can be measured as the area under the peak. In some embodiments, the signal intensity is measured as the area under the peak in an immunodetection.

In some embodiments, the one or more biomarker values are determined through performing a capillary electrophoresis under non-reducing conditions. Specifically, biomarker signals are detected by performing the capillary electrophoresis and immunodetection, and then the one or more biomarker values are determined based on the detected biomarker signals. According to certain preferred embodiments, the one or more biomarker values include IP₅₈, IP₁₃₀, IP₁₈₀, or a combination thereof. According to certain preferred embodiments, based on a ratio of said biomarker values selected from the group consisting of

$$\frac{I {P}_{130}}{I {P}_{58}} , \frac{I {P}_{180}}{I {P}_{58}} , and \frac{I {P}_{130} + I {P}_{180}}{I {P}_{58}} ,$$

the patient is assigned as having or not having HCC, OC or BC, or having or not having a change in HCC, OC or BC health state, or having or not having a risk of HCC, OC or BC.

The present invention is further illustrated by the following examples, which are provided for the purpose of demonstration rather than limitation.

### Examples

### 1. Materials and Methods

### 1.1 Clinical Samples

Pre-operation HCC, BC and OC plasma samples were provided by Dr. Ming-Chih Ho, Dr. Wen-Hong Kuo and Dr. Pao-Ling Torng from the National Taiwan University Hospital. Blood samples were treated with 0.5M EDTA and protease inhibitors before spinning in a benchtop centrifuge at 3000 RPM at 4° C. for 15 minutes. The supernatant was saved as the plasma fraction and stored at −80° C. until usage.

### 1.2 Western Blot Analysis

For each well, 0.3 μL of plasma sample was mixed with SDS-containing sample dye (0.04 M Tris-HCl pH 6.8, 1 M glycerol, 0.05 M SDS with bromophenol blue) and with or without 0.3 μL β-mercaptoethanol for reducing or non-reducing analyses, respectively. Following heating for 5 minutes, the sample mixture was loaded into wells in 4% stacking/12% separating Tris-based polyacrylamide gel. After SDS-PAGE, the gel was incubated with electrophoresis solution (0.025 M Tris, 0.2 M glycine, 3 mM SDS), and proteins were transferred to the nitrocellulose membrane in 0.02 M of 3-(cyclohexylamino)-1-propanesulfonic acid (CAPS) in 10% methanol, pH 11. After the electrotransfer, the membrane was subjected to immunoblotting with indicated antibodies. The blot was blocked with 1% BSA in TBST buffer (0.02 M Tris, 0.14 M NaCl, 0.1% Tween 20, pH 7.6), and then incubated with anti-A1AT (Abcam, ab207303, rabbit-derived, 1:5,000 diluted) overnight at 4° C. and anti-rabbit conjugated with horseradish peroxidase (HRP) (Jackson, donkey-derived, 711-035-152, diluted 1:10,000) for 1 hour at RT, with TBST wash for three times following each antibody incubation. Immediately after the overlay with the reagents for chemiluminescence analyses, the signals of the membrane were using LAS-4000 (Fujifilm, Japan).

### 1.3 Automated Capillary Electrophoresis-Immunodetection

The reagents and equipment were purchased from BioTechne, USA, unless specified otherwise. Plasma samples were diluted 1:200, and 5× Fluorescent Master Mix was added to each sample. Heating took place for 30 minutes in a water bath. In the sample plate, four microliters of each sample and electrophoresis buffer for separating proteins ranging from 12 to 230 kDa were loaded. Also, 1% bovine serum albumin (or antibody diluent, Bionovas, AA0530-0250), primary and secondary antibody solutions, chemiluminescence reagents, and wash buffer were utilized per the manufacturer's instructions. For the biotinylated SimpleWestern size standard, antibody diluent, and streptavidin-HRP (Genetex, GTX27403) were used to replace primary and secondary antibody solutions. The primary antibody was anti-A1AT (Abcam, ab207303, rabbit-derived, diluted 1:2,000) and anti-rabbit HRP conjugate (Jackson, donkey-derived, 711-035-152) was employed as the secondary antibody. After centrifuged for 5 min at 1000 g at room temperature, the plate, along with capillaries, was loaded into the SimpleWestern™ system (BioTechne USA) operated with Compass software (version 6.1.0). The mass range was set to be the standard 12- to 230-kDa protocol, and the separation time the default of 25 minutes. Using the fluorescently labeled protein standards, the chemiluminescence signals versus apparent MW were produced.

### 1.4 Migration Profile Analysis

The data with SimpleWestern system were analyzed using Microsoft Excel 2021 and its Visual Basic for Applications (VBA) package. Raw data obtained from the Compass for SW software were converted to text format, and then transformed into graphs, with the x-axis representing molecular weight and the y-axis representing the intensity of the immunodetection signals. To quantify the peak in a specific mass range, we employed an in-house peak detection and integration program, which was further validated through manual confirmation. The areas in particular mass ranges were then utilized to construct marker indices.

### 2. Results

2.1 Multimeric A1AT Species of 62-kDa, 135-160 kDa and 225-295 kDa Increased Relative to the 56-kDa Monomeric Form in the Plasma of Patients Diagnosed with Hepatocellular Carcinoma (HCC), Ovarian Cancer (OC), or Breast Cancer (BC)

The plasma samples from three healthy subjects and three HCC patients were subjected to conventional SDS-PAGE and western blotting under non-reducing conditions to compare the disulfide-mediated A1AT conjugates between these two groups. In addition to the 56-kDa monomeric species, there were three groups of A1AT species discerned based on their molecular sizes. The first was a 62-kDa species, migrating slightly slower than the A1AT monomer. The second 150-kDa group had two protein bands at 135 and 160 kDa. The third 260-kDa group had three polypeptides of molecular sizes of 225, 265 and 295 kDa. Western blot analyses revealed the increases in these three A1AT groups, which were documented using densitometric analyses. For healthy subjects, the signals were 0.21˜0.30 and 0.26˜0.38 for the 150- and 260-kDa groups, respectively. In contrast, the values increased up to 0.26˜0.36 and 0.34˜0.43 for HCC patients (data not shown). Similar findings could be seen for A1AT species from ovarian cancer (OC), or breast cancer (BC) (data not shown). As all three groups of multimeric structures disappeared in reducing SDS-PAGE (FIG. 1), disulfide bond is the putative linkage connecting A1AT subunits and other partners. Since there are many factors may interfere with accurate quantification based on conventional western blot analyses, we decided to use a different approach for the evaluation of these A1AT multimeric structures.

2.2 Peaks P₁₃₀ and P₁₈₀ with Capillary Electrophoresis Correspond to the 150- and 260-kDa Groups in SDS-PAGE Analyses

To better quantify how A1AT multimeric structures changed in cancer patients, we took advantage of an automated capillary electrophoresis-immunodetection method. This platform allowed us to observe these A1AT multimeric structures in the migration profiles for the tested plasma samples under non-reducing conditions. In the profiles for healthy subjects, three distinct peaks were observed, including P₅₈, P₁₃₀, and P₁₈₀. There were minimal differences in the overall pattern observed between women and men. The 62-kDa species likely becomes integrated into peak P₅₈ for monomeric A1AT, due to limited resolution power of capillary electrophoresis, and thus not measurable with this assay. The other two groups of A1AT species have been resolved into peaks P₁₃₀ and P₁₈₀. Based on their migratory positions, the former corresponded to the 150-kDa group, and the latter was for the 260-kDa one. To quantify these increases, we established three indices:

$$\frac{I {P}_{130}}{I {P}_{58}} , \frac{I {P}_{180}}{I {P}_{58}} and \frac{I {P}_{130} + I {P}_{180}}{I {P}_{58}} ,$$

where IP₅₈, IP₁₃₀ and IP₁₈₀ represent the peak areas of the respective species. The values of

$$\frac{I {P}_{130}}{I {P}_{58}} and \frac{I {P}_{180}}{I {P}_{58}}$$

were 0.02˜0.04 and 0.05˜0.11 for three healthy controls and 0.04˜0.11 and 0.13˜0.31 for HCC patients (data not shown). These findings were largely in line with the results of densitometric analyses (FIG. 1), strongly suggesting that the two groups of multimeric structures can be quantitatively analyzes using this automated system.
2.3 for Most HCC, BC and OC Patients, there were Notable Increases in these Two Peaks P₁₃₀, and P₁₈₀ in Comparison with Peak P₅₈

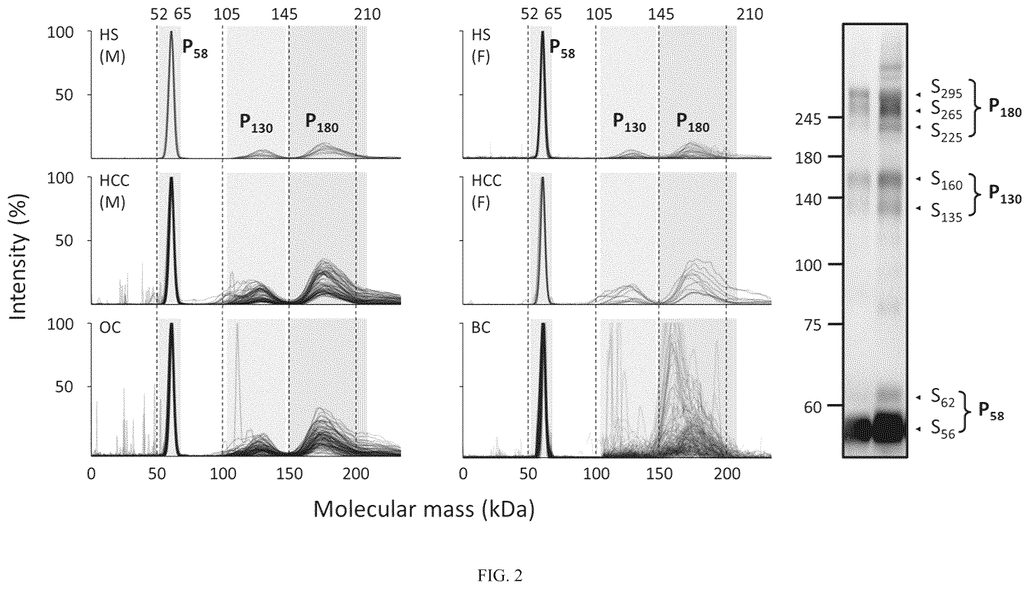

Samples from nine male and 28 female healthy controls were subjected to the automated capillary electrophoresis-immunodetection method, and we found gender factor has little effect on the migration profiles of A1AT multimeric structures (FIG. 2). Plasma samples from fifty-three male HCC, nine female HCC, 51° C. and 161 BC patients at stage 0 to 2 were subjected to the analyses using SimpleWestern system. These results showed that the protein species corresponding to P₁₃₀ and P₁₈₀ in the patient groups indeed had significant increases (FIG. 2). For BC patients, it seems that P₁₈₀ had a more prominent change than the other cancer groups but the increment in P₁₃₀ is not as consistent as the other two cancers (FIG. 2).

For

$$\frac{I {P}_{130}}{I {P}_{58}}$$

index, healthy subjects exhibited an average value of approximately 0.04. In contrast, male HCC, female HCC, OC and BC patients showed the means of 0.08, 0.12, 0.08 and 0.13, respectively. Regarding the

$$\frac{I {P}_{180}}{I {P}_{58}}$$

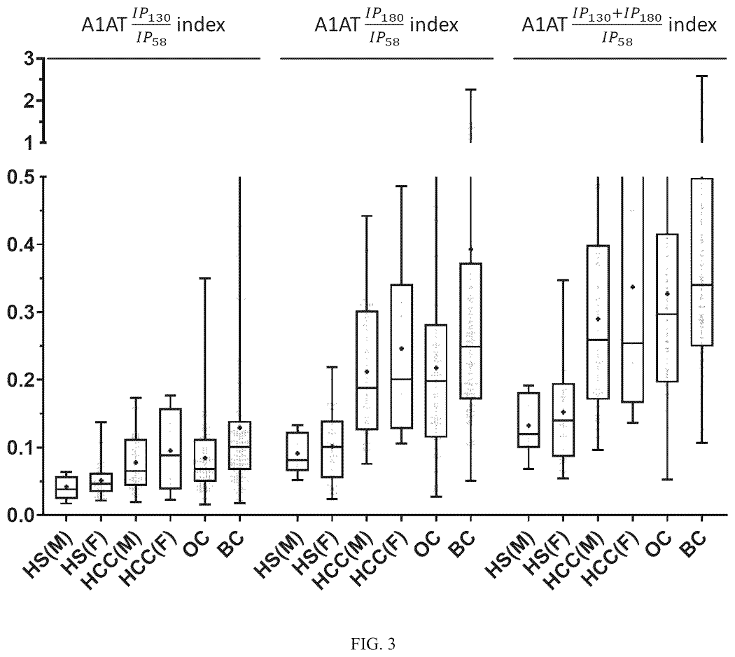

index, the healthy control group averaged about 0.1, while HCC, OC and BC patients displayed values in the range of 0.2 to 0.4 (FIG. 3). These results were consistent with the observed increases in the heights of peaks P₁₃₀ and P₁₈₀ in the migration profiles (FIG. 2). To consider the sum of changes in both P₁₃₀ and P₁₈₀ multimers, we introduced the

$$\frac{I {P}_{130} + I {P}_{180}}{I {P}_{58}}$$

index. Healthy controls had values ranging from 0.15˜0.16, which increased to 0.3˜0.35 for HCC patients, 0.33 for OC and 0.5 for BC group (FIG. 3). These all indicate a notable increase in peaks P₁₃₀ and P₁₈₀ for most HCC, OC and BC patients. The statistical parameters of

$$\frac{I {P}_{180}}{I {P}_{58}} and \frac{I {P}_{130} + I {P}_{180}}{I {P}_{58}}$$

and indices exhibited remarkable similarity, consistent with the observation that the increase in peak P₁₈₀ was much higher than peak P₁₃₀ in most migration profiles. Considering the distribution of these values across quartiles (FIG. 3), it is possible to establish cutoffs for distinguishing patients from healthy controls.
2.4 the A1AT Multimeric Indices Based on Peaks P₁₃₀ and P₁₈₀ Demonstrated Superior Performance in the Screening of HCC, OC, and BC Patients

We employed receiver operating characteristic (ROC) curves to assess the potential of these A1AT indices in distinguishing patients from healthy subjects. For

$$\frac{I {P}_{130}}{I {P}_{58}}$$

index, the curves were generally closer to the random classifier line for HCC and OC patient groups, but is closer to the ideal classifier for BC patients. The ROC curve for the

$$\frac{I {P}_{180}}{I {P}_{58}}$$

index exhibited a clearer separation from the random classifier line, allowing for sensitivity levels in the range of 77 to 100% at a cutoff value of 0.11 to 0.14. The distinction was the best for the BC patient group. The sensitivity of the

$$\frac{I {P}_{130} + I {P}_{180}}{I {P}_{58}}$$

index was around 66 to 70% with a cutoff of 0.14 to 0.20. While still performing the best for BC patients, this index could not outperform the

$$\frac{I {P}_{180}}{I {P}_{58}}$$

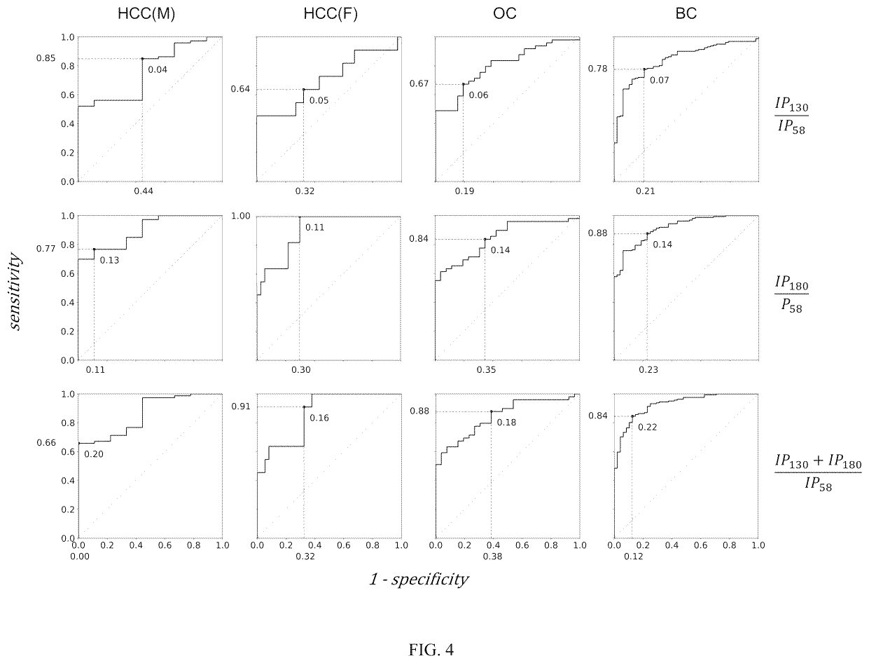

index much (FIG. 4).

We then explored the potential of A1AT indices in the screening of cancer patients. First, based on our ROC curve analyses (FIG. 4), we selected

$$\frac{I {P}_{180}}{I {P}_{58}} and \frac{I {P}_{130} + I {P}_{180}}{I {P}_{58}}$$

to test how sensitive they were in detection of cancer patients. Second, in clinical practice, a single test can have only one set of criteria for different types of cancers. With these considerations, we tentatively set the cutoff for

$$A 1 AT \frac{I {P}_{180}}{I {P}_{58}}$$

at 0.13 and that for

$$\frac{I {P}_{130} + I {P}_{180}}{I {P}_{58}}$$

### was 0.19. Overall, the use of

$$A 1 AT \frac{I {P}_{180}}{I {P}_{58}} and \frac{I {P}_{130} + I {P}_{180}}{I {P}_{58}}$$

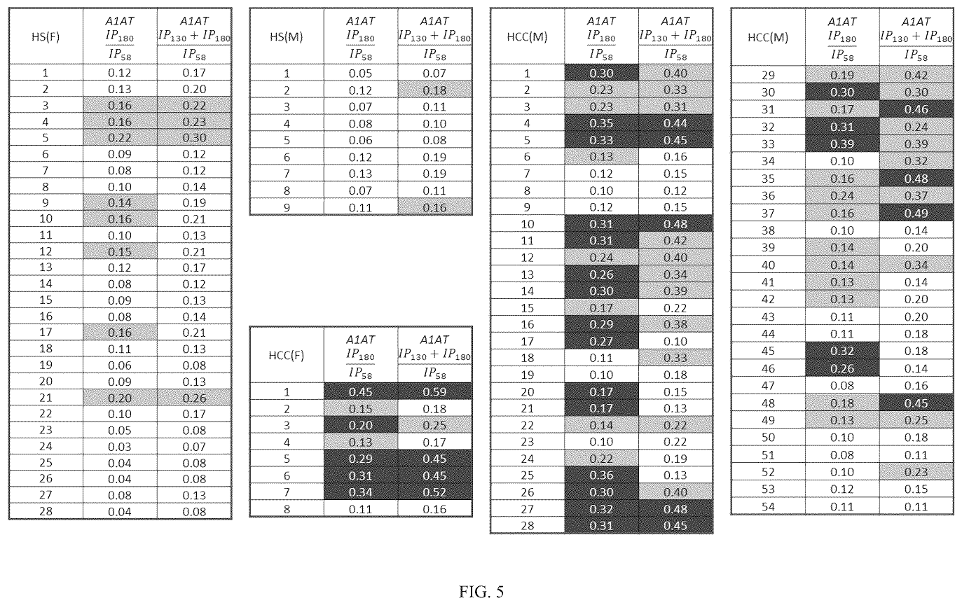

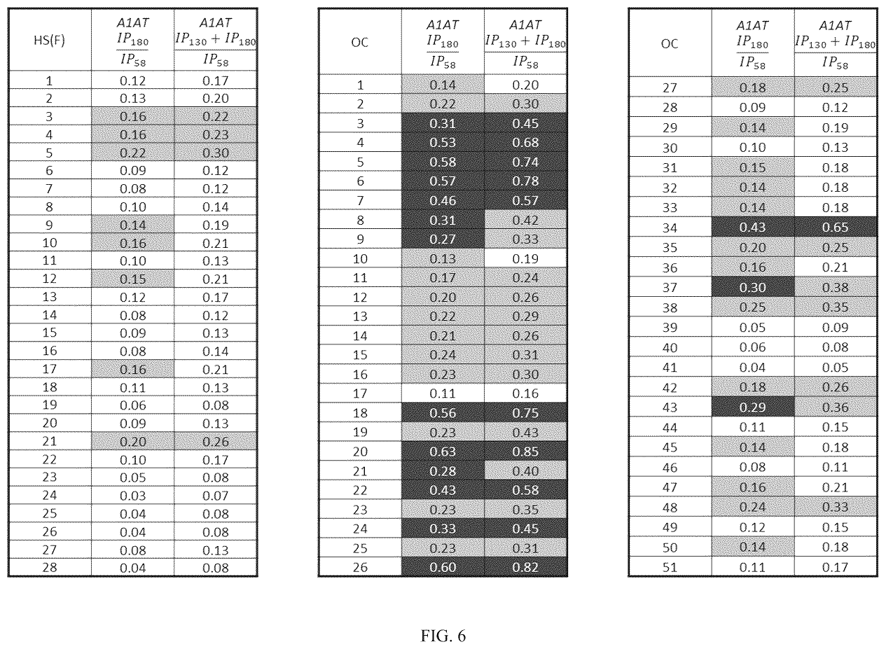

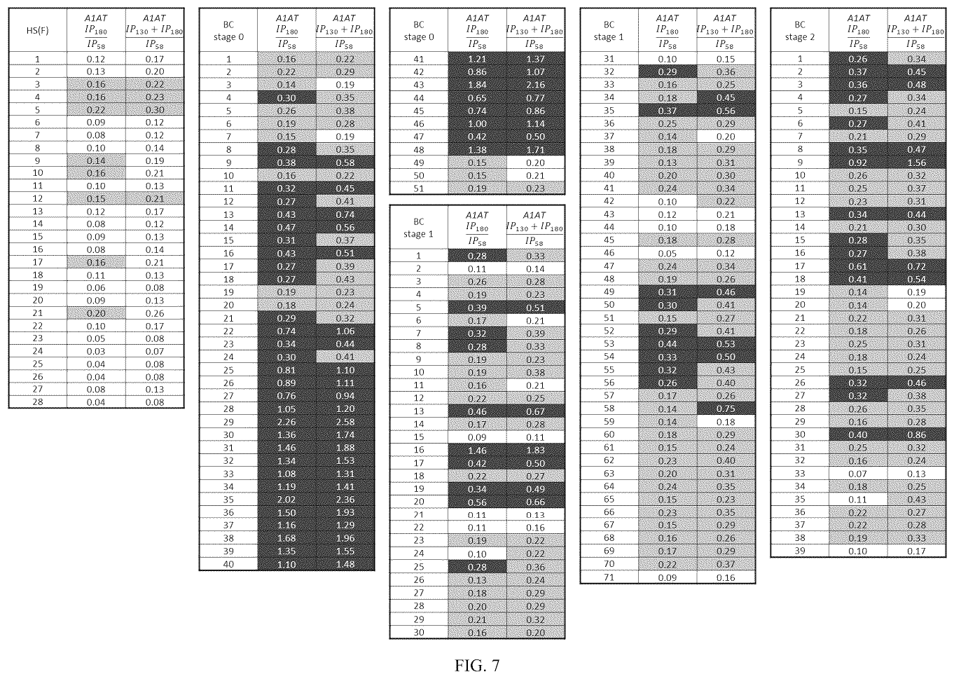

indices could identify 82, 80, and 94% of HCC, OC, and BC patients, respectively (FIGS. 5 to 7). In these cancer patient groups, there were more patients showing values two times higher than the cutoff of

$$\frac{I {P}_{180}}{I {P}_{58}} than \frac{I {P}_{130} + I {P}_{180}}{I {P}_{58}} ,$$

consistent with the separation results in ROC analyses. Also, there were more patients showing higher

$$\frac{I {P}_{180}}{I {P}_{58}}$$

values for BC patients than HCC or OC patients. Altogether, out results indicate that these indices offer excellent sensitivity and specificity in detecting cancer patients.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

1. A method for diagnosing a cancer health state in a patient, comprising:
determining, in a plasma sample from said patient, one or more biomarker values that correspond to alpha-1 antitrypsin (A1AT)-containing complex structures; and
assigning the patient as having or not having cancer, or having or not having a change in cancer health state, or having or not having a risk of cancer based on said biomarker values,
wherein said cancer is selected from the group consisting of hepatocellular carcinoma (HCC), ovarian cancer (OC), and breast cancer (BC).

2. The method of claim 1, wherein determining the biomarker values comprises performing an in vitro assay, wherein said in vitro assay comprises a capture reagent for A1AT or an A1AT-containing complex structure.

3. The method of claim 2, wherein the capture reagent is an antibody.

4. The method of claim 2, wherein the in vitro assay is a capillary electrophoresis under non-reducing conditions.

5. The method of claim 2, wherein the one or more biomarker values include IP₅₈, IP₁₃₀, IP₁₈₀, or a combination thereof.

6. The method of claim 1, wherein the assigning is based on a ratio of said biomarker values selected from the group consisting of
$$\frac{I {P}_{130}}{I {P}_{58}} , \frac{I {P}_{180}}{I {P}_{58}} , and \frac{I {P}_{130} + I {P}_{180}}{I {P}_{58}} .$$

7. A method for diagnosing a risk of the change or presence of a cancer in a patient, comprising:
determining, in a plasma sample from said patient, one or more biomarker values that correspond to alpha-1 antitrypsin (A1AT)-containing complex structures; and
assigning the patient as having or not having cancer, or having or not having a change in cancer health state, or having or not having a risk of cancer based on said biomarker values,
wherein said cancer is selected from the group consisting of hepatocellular carcinoma (HCC), ovarian cancer (OC), and breast cancer (BC).

8. The method of claim 7, wherein determining the biomarker values comprises performing an in vitro assay, wherein said in vitro assay comprises a capture reagent for A1AT or an A1AT-containing complex structure.

9. The method of claim 8, wherein the capture reagent is an antibody.

10. The method of claim 8, wherein the in vitro assay is a capillary electrophoresis under non-reducing conditions.

11. The method of claim 7, wherein the one or more biomarker values based on IP₅₈, IP₁₃₀, IP₁₈₀, or a combination thereof.

12. The method of claim 7, wherein the assigning is based on a ratio of said biomarker values selected from the group consisting of
$$\frac{I {P}_{130}}{I {P}_{58}} , \frac{I {P}_{180}}{I {P}_{58}} , and \frac{I {P}_{130} + I {P}_{180}}{I {P}_{58}} .$$

13. A kit for performing a method for diagnosing a cancer health state in a patient, comprising a capture reagent for A1AT or an A1AT-containing complex structure, and instructions for performing the method, wherein said cancer is selected from the group consisting of hepatocellular carcinoma (HCC), ovarian cancer (OC), and breast cancer (BC).

14. The kit of claim 13 wherein the method comprising determining one or more biomarker values that correspond to A1AT-containing complex structures using the capture reagent, and assigning the patient as having or not having cancer, or having or not having a change in cancer health state, or having or not having a risk of cancer based on said biomarker values.

15. The kit of claim 13, wherein the capture reagent is an antibody.

16. The kit of claim 14, wherein the capture reagent is an antibody.

17. The kit of claim 14, wherein determining the biomarker values comprises performing an in vitro assay using the capture reagent.

18. The kit of claim 17, wherein the in vitro assay is a capillary electrophoresis under non-reducing conditions.

19. The kit of claim 14, wherein the one or more biomarker values include IP₅₈, IP₁₃₀, IP₁₈₀, or a combination thereof.

20. The kit of claim 14, wherein the assigning is based on a ratio of said biomarker values selected from the group consisting of
$$\frac{I {P}_{130}}{I {P}_{58}} , \frac{I {P}_{180}}{I {P}_{58}} , and \frac{I {P}_{130} + I {P}_{180}}{I {P}_{58}} .$$
